(19) United States
(12) Patent Application Publication
(10) Pub. No.: US 2006/0204416 A1
(43) Pub. Date: Sep. 14, 2006

(54) Toothbrush sanitizer

(76) Inventors: John E. Hayes, Cerritos, CA (US); Songlin Shao, Hacienda Hts., CA (US)

Correspondence Address: JOHN E. HAYES, 11104 HIBBING ST., CERRITOS, CA, 90703, US

(21) Appl. No.: 11/076,829

(22) Filed: Mar. 10, 2005

(51) Int. Cl. A61L 2/18

(52) U.S. Cl. 422/300; 422/28

(57) ABSTRACT

The toothbrush sanitizer is designed to sanitize and store one or two (maximum) toothbrush(es) in a clean and sanitized, closed environment. The toothbrushes are sanitized using a simple, common household product, MOUTHWASH. This toothbrush sanitizer is constructed of a durable plastic and is designed with two side-by-side vertical compartments and two reservoirs at the bottom of each compartment. The reservoirs are separated from the compartments by screens. These screens allow the mouthwash to pass through from the reservoirs and saturate the bristles of the toothbrushes, and, they also serve as a platform for the toothbrushes to rest on so that the mouthwash can drain from the bristles of the toothbrush back into the reservoirs. The compartments are separated by a permanent wall extending from the bottom of the unit to the rim. Both compartments are covered by a single dome lid. The overall design of the toothbrush sanitizer is such that the toothbrushes are sanitized by maneuvering and/or shaking the unit in such a way as to bathe the bristles of the toothbrushes with the mouthwash, hence, killing and/or inhibiting, the growth of harmful bacteria or other harmful pathogens. The screens that separate the reservoirs (which hold the mouthwash) from the compartments (which hold the toothbrushes) are removable so that the unit can be easily cleaned. The evaporative property of the mouthwash is what maintains the sanitized environment within the enclosed unit.

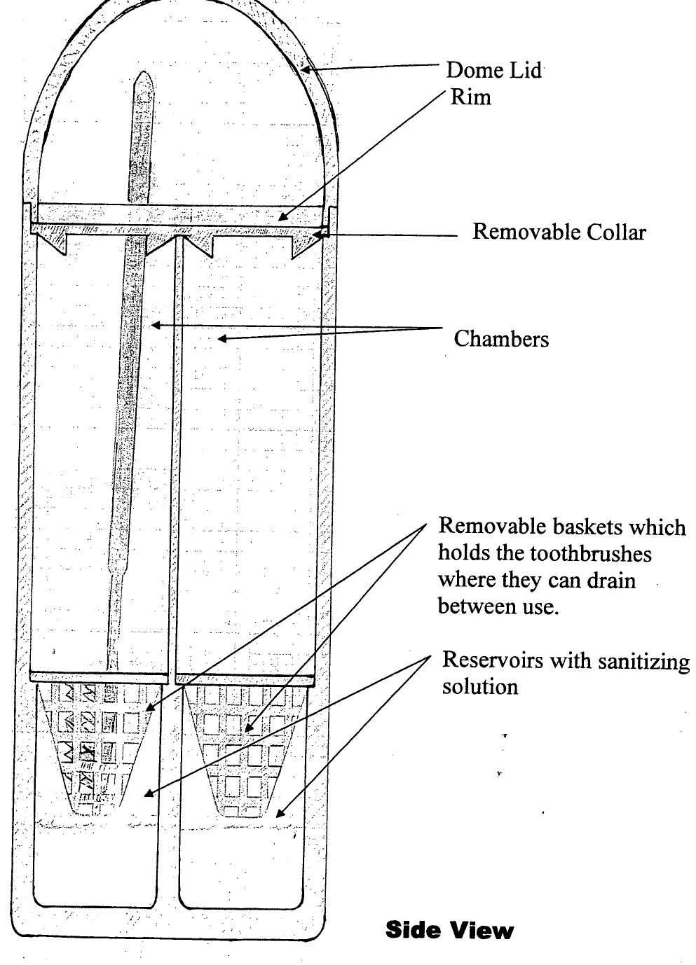

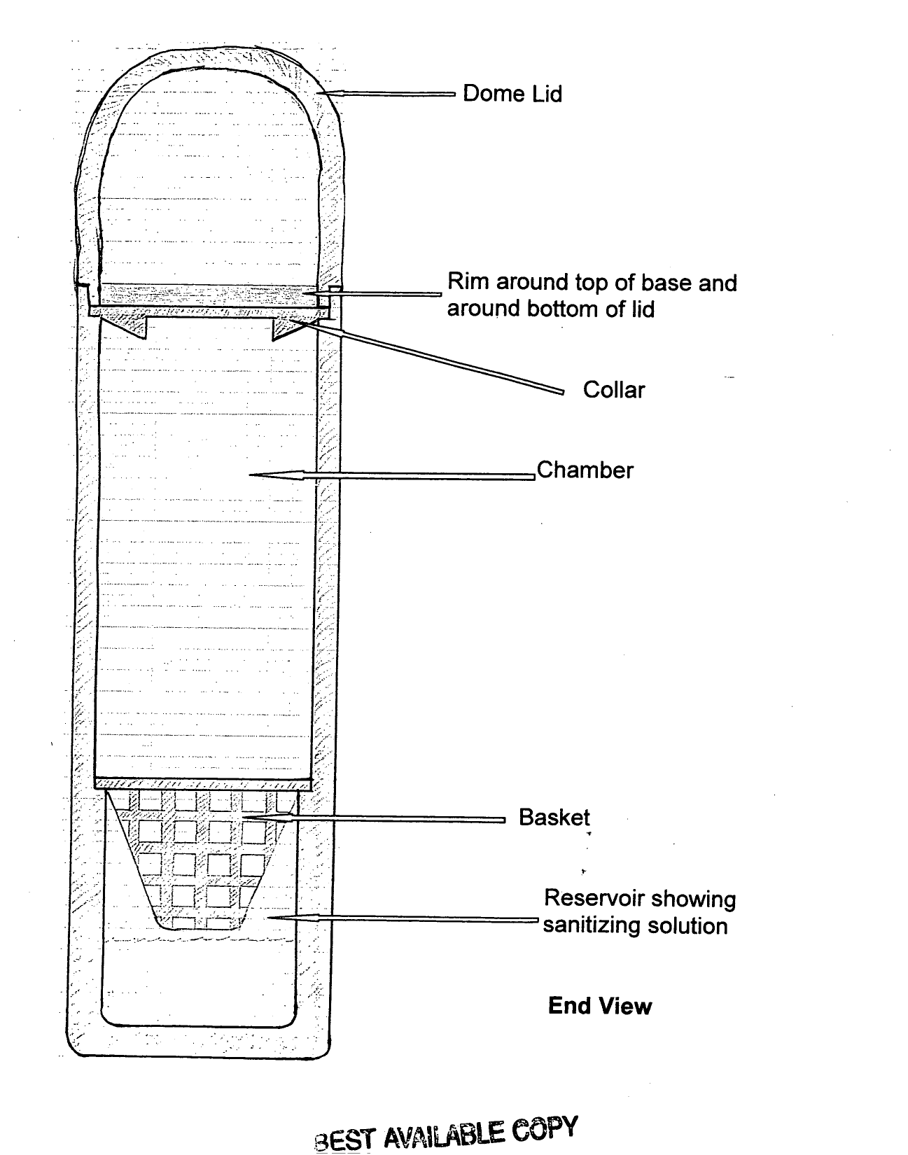

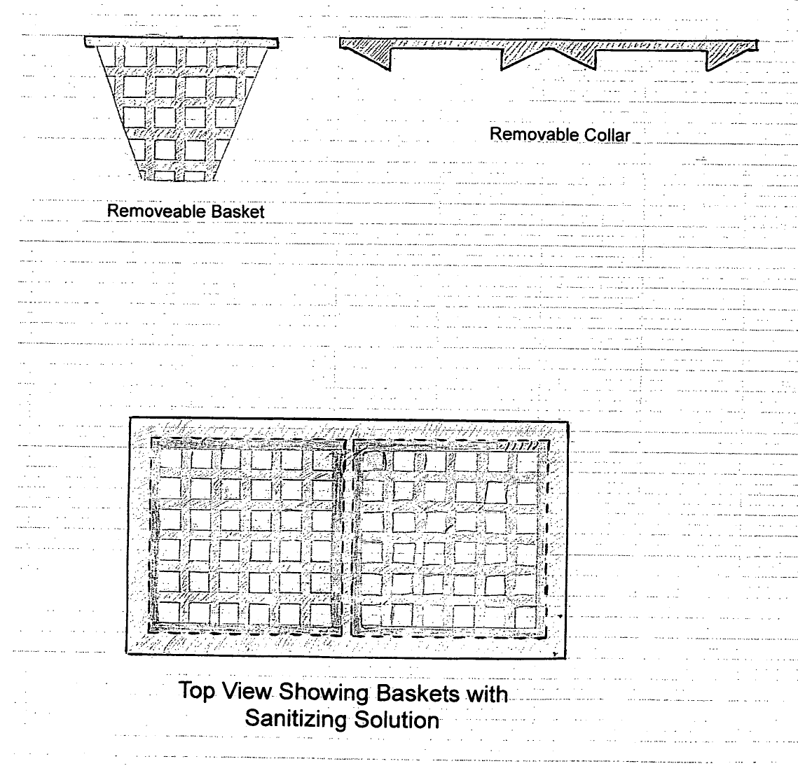

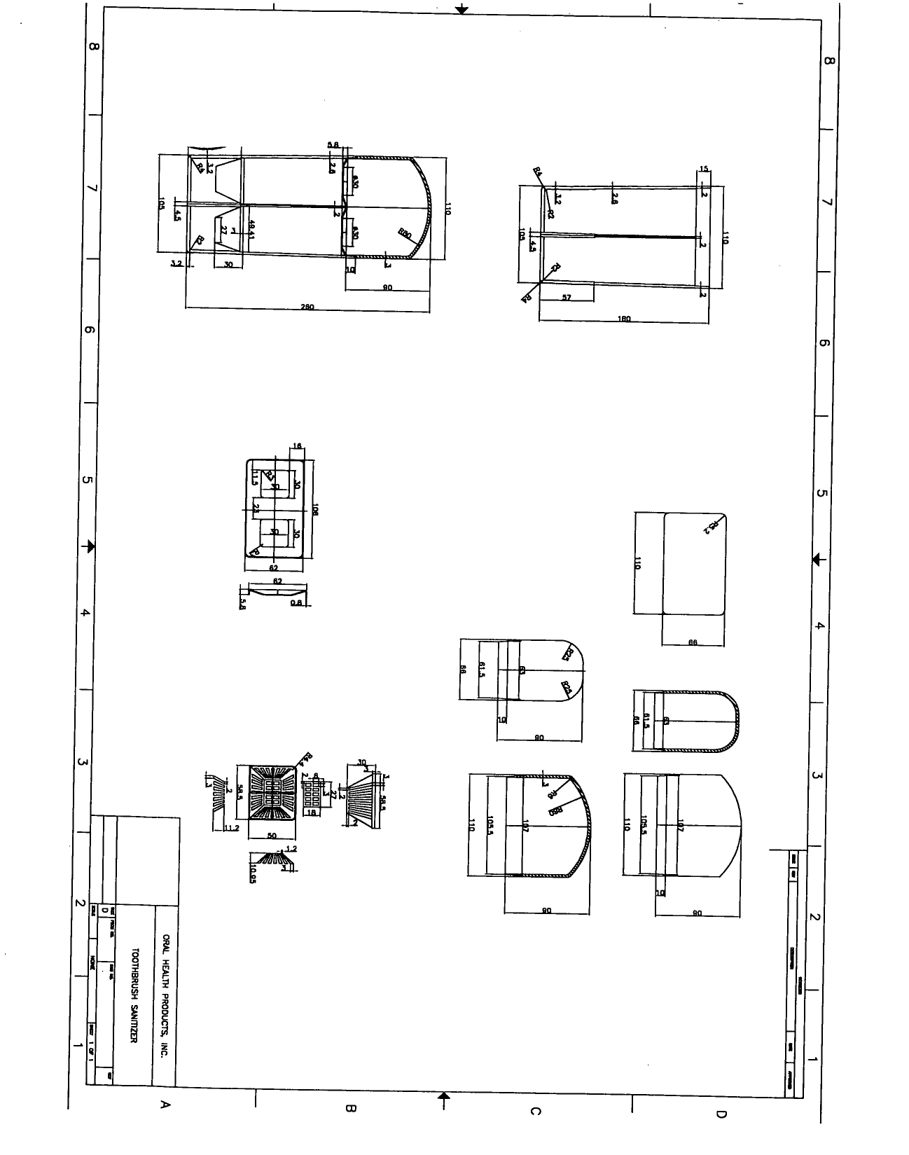

### SUMMARY

This toothbrush sanitizer is easy to use, it is safe and effective, and, it is economical.

This toothbrush sanitizer is designed to utilize one's own mouthwash for sanitization. It does not require caustic chemicals, any form of electricity, hot water and/or steam, ultraviolet light or radiation or anything that could be considered a health hazard or dangerous. This toothbrush sanitizer is constructed of a durable plastic with removable screens for easy cleaning. Each toothbrush sanitizer is designed with two vertical compartments, two reservoirs at the bottom of the compartments, two screens separating the reservoirs from the compartments, a vertical wall dividing the unit into two sides, one reservoir and one compartment on each side and a single dome lid that covers the two compartments. Because of the unique design of the unit, it utilizes the evaporative property of the mouthwash to maintain a sanitized environment within the unit making it ideal for storing toothbrushes between each use.

What is claimed is:

1. This toothbrush sanitization unit sanitizes one or two toothbrushes using a common, everyday household product, MOUTHWASH.
This toothbrush sanitization unit is designed with two vertical compartments, two reservoirs, one each at the bottom of each compartment, two screens, each separating a compartment from a reservoir, a wall that extends from the bottom of the unit to the rim dividing the unit into two halves. Each half of the unit contains one compartment and one reservoir. The two compartments are covered by a single dome lid.
This toothbrush sanitization unit, because of its unique design and the evaporative property of the mouthwash, maintains a sanitized environment. The mouthwash kills harmful bacteria and other pathogens as well as inhibits the growth of harmful bacteria and other pathogens.
This toothbrush sanitization unit is so designed that it does not require the use of any form of electricity, hot water or steam, ultraviolet light or radiation, ozone or ionization or any harmful chemicals to eliminate harmful pathogens.
This toothbrush sanitization unit is designed to be safe, effective, and easy to use as well as economical.
This toothbrush sanitization unit is so designed that the toothbrushes can be sanitized and then stored in the unit between uses. The unique screens allow the mouthwash to pass from the reservoirs to saturate the bristles of the toothbrushes and then become platforms to allow the mouthwash to drain from the bristles back into the reservoirs.
